(19) United States
(12) Patent Application Publication
(10) Pub. No.: US 2025/0366806 A1
(43) Pub. Date: Dec. 4, 2025

(54) GANTRY APPARATUS FOR A CT SYSTEM

(71) Applicant: Siemens Healthineers AG, Forchheim (DE)

(72) Inventors: Elmar GARCIA, Nuernberg (DE); Hans-Juergen MUELLER, Pretzfeld (DE); Christoph DICKMANN, Nuernberg (DE); Christian HOFMANN, Erlangen (DE)

(73) Assignee: Siemens Healthineers AG, Forchheim (DE)

(21) Appl. No.: 19/225,414

(22) Filed: Jun. 2, 2025

(30) Foreign Application Priority Data
Jun. 3, 2024 (DE) 10 2024 205 082.2

(51) Int. Cl. A61B 6/00; G16H 30/20; G16H 40/67

(52) U.S. Cl. CPC A61B 6/4447; A61B 6/4405; A61B 6/4458; G16H 30/20; G16H 40/67; A61B 2560/0214

(57) ABSTRACT

A gantry apparatus for a CT system comprises a gantry and a movement system. The gantry includes a plurality of radiation sources and radiation detectors which are each arranged on a plane around a main axis. The gantry is mounted to move laterally via the movement system. The movement system has guide elements and is configured such that the gantry is movable in a straight line parallel to the main axis via the guide elements.

### CROSS-REFERENCE TO RELATED APPLICATION(S)

The present application claims priority under 35 U.S.C. § 119 to Germany Patent Application No. 10 2024 205 082.2, filed Jun. 3, 2024, the entire contents of which is incorporated herein by reference.

### FIELD

One or more example embodiments of the present invention relate to a gantry apparatus for a CT system and a CT system.

### BACKGROUND

Computer tomography (abbreviation: “CT”) is an established imaging method in medicine. In a CT examination, a person or generally speaking, an object to be examined, is pushed on a table into the recording component of a CT system which then rotates around the object to be recorded and produces a multiplicity of images which are then processed into a three-dimensional image stack according to established methods.

In some medical examinations or applications, it may be advantageous if the gantry of a CT system can be moved towards and away from a patient, or sideways (from the right or the left or from the side of the head or the feet) over the patient, e.g. for an interventional CT or a CT on an operating table. As a result, the examination can be performed with greater ease, speed and effectiveness.

The gantry can be moved, for example, via a trolley. However, the design must be such that the tube as well as the detector module (DMS) can still be rotated around the patient in order to record complete image data. Until now, this has usually meant complicated, heavy and expensive technical solutions for the implementation of functions. In addition, precise adjustment of such a mobile gantry along its axis of rotation is quite complicated. A correct recording position can sometimes only be achieved after repeated maneuvering.

Systems currently in use have only very limited mobility, if any, in particular when it comes to being used on different tables and in different rooms. Another problem is limited patient accessibility, either because not every table or not all possible patient positioning can be used. Many systems have no gantry opening or one that is too small. In addition, it is currently not possible to simply record volume ranges along the axis of rotation.

### SUMMARY

It is an object of one or more example embodiments of the present invention to provide a gantry apparatus for a CT system and a CT system with which the disadvantages described above are avoided.

At least this object is achieved by a gantry apparatus and a CT system as claimed in the independent claims and the dependent claims.

A gantry apparatus, according to embodiments of the present invention, for a CT system comprises a gantry and a movement system:
the gantry comprising a plurality of radiation sources and radiation detectors which are each arranged on a plane around a main axis, and the gantry being mounted in such a way that it can be moved laterally via the movement system,
the movement system having guide elements and being designed such that the gantry can be moved in a straight line parallel to its main axis via the guide elements.

The gantry comprises the components responsible for image acquisition, that is to say, the radiation sources and the detector arrangement. In contrast to a conventional, rotating gantry, which generally has a single radiation source and a single radiation detector and must therefore rotate, the gantry, according to embodiments of the present invention, comprises a plurality of radiation sources and radiation detectors. These are arranged in such a way that images can be recorded with pairs of radiation sources and radiation detectors (or groups of radiation detectors). These elements do not necessarily have to be static (which is a preferred embodiment, however), but can be movable in order to be able to serve a certain recording angle range (which is another preferred embodiment).

The term “plurality” is to be understood here as at least two, with significantly more than two being preferable. The radiation detectors can, for example, form a closed ring which can be read out area by area. The granularity of this ring during a readout can then be understood as individual radiation detectors of which several are combined into a group for image acquisition. In the case of a preferred gantry, the radiation source element has at least 10 radiation sources, in particular at least 36 radiation sources. It can also comprise 100 or more radiation sources.

For example, if there are 36 radiation sources, images with viewing angle differences of 10° each can be recorded. If these radiation sources can now be rotated by 10° around the main axis (corresponds to the axis of rotation in a conventional CT gantry), images can then be recorded from many more viewing angles. The more radiation sources there are, the less movement is required to produce sufficient images. The individual radiation sources can preferably be controlled individually.

The radiation sources are preferably arranged at regular intervals. This has the advantage that images can be taken from a multiplicity of recording angles without having to rotate the radiation source element.

The radiation sources are preferably Nanotube Field Emitters. These are small, inexpensive and can emit X-rays with an intensity suitable for examinations.

In general, it should be noted that the radiation source used for image acquisition and a radiation detector or radiation detector group used for this purpose are located opposite each other in the gantry.

The gantry apparatus need not necessarily have as many radiation detectors as there are radiation sources. There may well be more or fewer. However, a radiation detector (or a radiation detector group) should be large enough to be able to cover the beam cone of a radiation source or to cover the desired recording area.

Radiation detectors and radiation sources are each arranged on a plane around a main axis. The plane on which the radiation detectors are arranged and the plane on which the radiation sources are arranged can be located at the same positions, or they can be slightly offset relative to each other along the main axis of the CT system. If, for example, there is a complete detector ring enclosing 360°, it is advantageous if the planes are offset relative to each other so that the radiation sources do not have to radiate through a radiation detector onto an object to be captured or radiation sources are located in front of the radiation detector.

It is preferable that radiation sources and radiation detectors are arranged concentrically around the main axis. The main axis is the axis which runs through the center of the gantry and is parallel to the normal vector of the surface on which the gantry lies, i.e., as mentioned, it corresponds to the axis of rotation of a conventional gantry.

The gantry is (mechanically) mounted in such a way that it can be moved laterally via the movement system in order to adjust or set the optimum recording position and to record volume ranges. This means that it can be moved at least in a straight line parallel to its main axis, but possibly also orthogonal to its main axis. To this end, the movement system has guide elements, for example rails, on which the gantry can be moved in a straight line. The gantry can be mounted on the rails via rollers, for example.

A CT system, according to embodiments of the present invention, comprises a gantry apparatus according to embodiments of the present invention. In addition to the gantry, it can also have a control facility for controlling images as well as calculation and display units for calculating and displaying captured CT images.

Further, particularly advantageous embodiments and developments of the present invention will emerge from the dependent claims and the following description, it also being possible for the claims of one claim category to be developed analogously to the claims and parts of the description of another claim category and in particular, it also being possible for individual features of different exemplary embodiments or variants to be combined to form new exemplary embodiments or variants.

A preferred gantry apparatus is characterized in that the movement system has at least two rails as guide elements, which are arranged parallel to each other. Furthermore, it comprises movement elements which are connected to the gantry and are designed to move along the rails. This movement can preferably be of a sliding nature (via slide bearings) and/or via rollers and/or via balls. Alternatively or additionally, the movement system can preferably have linear ball bearings with or in which the gantry can slide.

According to a preferred embodiment, the guide elements can be arranged in a fixed position in the examination room, for example on the floor, on a wall or on the ceiling of the examination room.

Preferably, the movement system has a number of motors and is designed to move the gantry via a motor along the main axis and/or to rotate around the main axis. For example, a motor can drive wheels of the gantry on a rail or a linear motor can push the gantry, for example in a linear ball bearing or mounted on parallel rods via linear ball bearings. The gantry can also have wheels which slide in grooves (as guide elements).

It is not absolutely necessary for the gantry to rotate in order to capture images from many different recording angles. It is basically sufficient if the radiation sources and/or the radiation detectors can rotate around the main axis. This can be achieved in particular with a movement mechanism in the gantry. Alternatively, the radiation sources and/or the radiation detectors can be controlled individually along the circumference of the gantry, for example in a sequence following the circumference or in another predetermined sequence. As a result, a series of projection images of the examination object can be generated, which ensures sufficient coverage of the examination object and enables the reconstruction of tomographic image data sets such as those obtained in circular scanning of the examination object in a system with a rotating gantry.

Rotation around the main axis preferably takes place at an angle of less than 180° and is used to produce images from different viewing angles. It is preferable that with N radiation sources arranged in a circle at regular intervals, the movement takes place over an angle of 720°/N maximum, preferably 360°/N maximum.

A preferred gantry apparatus is characterized in that the radiation sources and radiation detectors of the radiation detector arrangement are arranged in a ring around the main axis. The gantry is then preferably designed to generate beams from several spatial directions through its center onto the radiation detectors via the radiation sources. At least the radiation sources can be rotatable around the main axis, but particularly preferably only by an angular range of less than 180°. According to an alternative embodiment, the radiation sources and/or the radiation detectors are arranged statically in the gantry apparatus. As a result, a rotation mechanism can be dispensed with and the gantry can be very narrow in design.

A preferred gantry apparatus is characterized in that the gantry has an open, for example C-shaped, ring or a closed ring which can be opened. For example, the closed ring can have a movable ring segment which can be removed, pivoted or moved to enable an opening along the circumference of the ring. This is highly advantageous for lateral approaches to the table, for example to position the ring in an examination position around the examination object. The opening of the gantry is preferably greater than 60 cm, particularly greater than 80 cm, so that the gantry can easily be slid over a person from the right or left. The fact that rotation of the gantry can basically be dispensed with makes this very easy. For example, a part of the ring of the gantry can be designed to swivel, this part preferably also comprising radiation sources and radiation detectors and cables to these being run from the side into the part with which it is connected to the rest of the ring (possibly via a hinge). Even if the radiation detectors and/or radiation sources are movable over a certain angular range (less than 180°), such a hinged gantry can be realized.

A preferred gantry apparatus comprises a robotic holding apparatus designed to be able to move the gantry along non-linear trajectories in the room. Such a robotic holding apparatus is preferably equipped with parallel or serial kinematics and preferably comprises a robotic arm or a hexapod.

A preferred gantry apparatus has intrinsic X-ray protection, preferably in the form of a lead lining of the gantry or subsections of the gantry.

A preferred gantry apparatus comprises interfaces for the integration of image and command transfer for external devices, in particular for optical tracking, external software for image processing or an image display in the room.

A preferred gantry apparatus comprises control elements for users, for example manual control or a data interface for control commands from a mobile device such as, for example, a tablet computer.

A preferred gantry apparatus is characterized in that the movement system has a swivel joint to enable tilting of the gantry about an axis orthogonal to its main axis, preferably via a motor. The main axis of the gantry can be tilted with ease via a swivel joint. Preferably, the swivel joint engages with the guide elements so that the gantry can continue to be moved parallel to its main axis even after rotation. The swivel joint enables a gantry to be rotated from a position for examining a recumbent patient to an examination of a standing patient.

A preferred gantry apparatus comprises a handle with power sensors for power-assisted movement of the gantry. This means that the gantry can also be moved manually, if necessary with motorized support.

A preferred gantry apparatus comprises a collision sensor, the gantry apparatus being designed particularly preferably and for sensor-supported collision avoidance or positioning. A collision sensor prevents collisions of the gantry during its movement. This serves to protect the gantry and patients.

A preferred gantry apparatus is characterized in that the movement system is additionally designed to move the gantry in a direction orthogonal to its main axis, preferably via a motor. As a result, the gantry can be adjusted even more precisely to an optimal examination position and accommodate volume ranges with ease. The movement system can also have guide elements for such movements.

A preferred gantry apparatus comprises a trolley, preferably an omnidirectional trolley. With such a trolley, it can be moved to the location of the next examination with ease. The movement system of the gantry is used for fine adjustment after positioning and for accommodating volume ranges. The trolley can preferably be locked for an examination. This prevents the gantry from moving unintentionally during an examination. Particularly preferably, the trolley comprises a support apparatus for the gantry.

A preferred gantry apparatus comprises a (preferably rechargeable) battery system for supplying energy to the gantry and preferably also to motors.

A preferred gantry apparatus comprises a radio system for wireless transmission of image data.

One or more example embodiments of the present invention solve several problems of X-ray based CT imaging, in particular during interventions such as, for example, minimally invasive or catheter-based interventions or percutaneous punctures, and operations such as, for example minimally invasive or open surgical interventions. A high quality image can be achieved, in particular a high spatial and temporal resolution as well as an arbitrarily selectable length for 3D volumes. This can be achieved by a high number of radiation sources and radiation detectors which can be moved by a certain angular range if necessary.

As the system is very light and also comparatively slim in design and can easily be provided with an opening, it enables very good patient accessibility, which is highly advantageous for surgeons and anesthetists in particular. A mobile and movable system is independent of the patient tables and patient positioning used. It also enables simple and safe operation without changing the patient position or device access points.

### BRIEF DESCRIPTION OF THE DRAWINGS

The present invention is explained again in more detail hereinafter with reference to the attached figures on the basis of exemplary embodiments. The same components are marked with identical reference numbers in the various figures. The figures are generally not to scale. The figures show:

FIG. 1A rough diagrammatic view of a CT system according to the prior art,

FIG. 2A gantry apparatus according to embodiments of the present invention viewed from the main axis,

FIG. 3A gantry apparatus according to embodiments of the present invention viewed from the side of the main axis,

FIG. 4A gantry apparatus on a robotic arm,

FIG. 5A gantry apparatus on a trolley,

FIG. 6A rotatable gantry apparatus on a trolley,

FIG. 7A gantry on rails.

### DETAILED DESCRIPTION

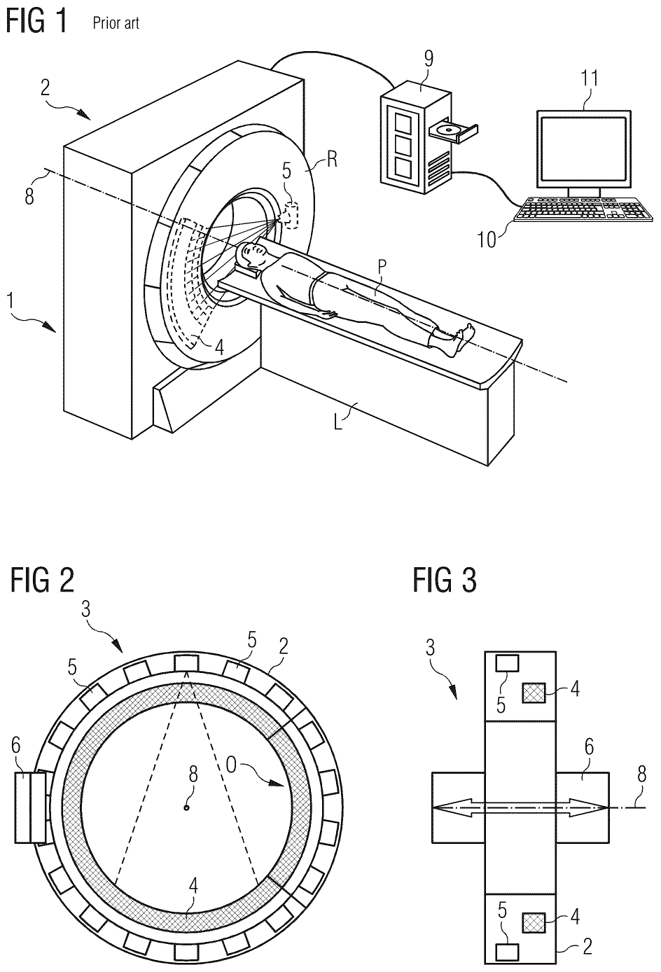

FIG. 1 shows a computer tomography system (CT system) 1 with a radiation detector 4 and a radiation source 5. The radiation source 5 is designed to expose the radiation detector 4 to radiation. The CT system 1 shown comprises a gantry 2 with a rotor 3. The rotor 3 comprises an X-ray source 5 as the radiation source 5, and the radiation detector 4, which is designed to detect X-rays.

The rotor 3 can be rotated around the axis of rotation 8. The patient P is supported on the patient table L and can be moved along the axis of rotation 8 through the gantry 2. The computing unit 9 is provided to control the CT system 1 and/or to generate an image data set based on signals detected by the radiation detector 4.

A (raw) X-ray image data set of the patient P is usually recorded from a multiplicity of angle directions via the radiation detector 4. A (final) image data set can then be reconstructed based on the (raw) X-ray image data set via a mathematical method, for example comprising a filtered back projection or an iterative reconstruction method.

The computing unit 9 serves here as a control facility 9 for controlling the CT system 1. An input facility 10 and an output facility 11 are connected to this computing unit 9. The input facility 10 and the output facility 11 can, for example, enable interaction by a user or the display of a generated image data set B.

FIG. 2 shows a gantry apparatus 3, according to embodiments of the present invention, viewed from the main axis. It comprises a gantry 2 and a movement system 6, which is shown from the side here as it is intended to enable movement parallel to the main axis 8. The gantry 2 here comprises eighteen radiation sources 5 and a ring of radiation detectors 4. The radiation sources 5 and the radiation detectors 4 of the radiation detector arrangement 4 are arranged in a ring around the main axis in the form of two concentric rings. The gantry 2 is designed to generate beams from several spatial directions through its center onto the radiation detectors 4 via the radiation sources 5. A beam cone is indicated with dashed lines here. The group of radiation detectors 4 which are illuminated by the beam cone can be used for recording.

The gantry 2 is designed as a closed ring which can be opened on the right side. This opening O of the gantry 2 should be greater than 60 cm. In this case, the radiation sources 5 and the radiation detectors 4 are arranged statically in the gantry apparatus 3. However, they could also be rotatable by 20° around the main axis in order to capture images from several angles.

FIG. 3 shows the gantry apparatus 3 from FIG. 2 viewed from the side of the main axis 8. It can be seen here that the radiation sources 5 arranged in a ring lie on one plane and the ring of radiation detectors 4 lie on another plane, offset in relation to it, so that the radiation detectors 4 do not cover any radiation sources 5. The movement system 6 has guide elements S (not visible here) and is designed in such a way that the gantry 2 can be moved in a straight line parallel to its main axis 8 (in the direction of the double arrow) via the guide elements S.

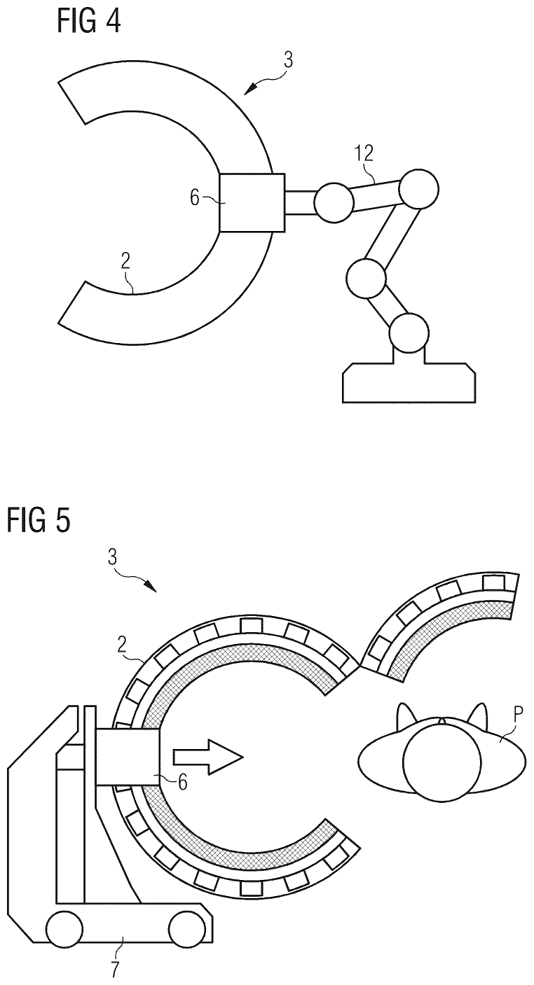

FIG. 4 shows a gantry apparatus on a robotic arm 12, which is an example of a robotic holding apparatus and is designed to be able to move the gantry 2 along non-linear trajectories in the room. The gantry 2 is open in this example and could only take images within a limited angular range in a static setup or take 360° images in a setup which can rotate +/−30° about the main axis 8. It is not necessary for the entire gantry 2 to rotate, it is sufficient if radiation sources 5 and radiation detectors 4 can rotate.

FIG. 5 shows a gantry apparatus 3 on a trolley 7. This can be, for example, an omnidirectional trolley 7 which can preferably be locked for an examination. In this example, the gantry 2 can be opened to be moved sideways over a patient P (see arrow). The illustration shows the open gantry 2. In the examination position, the ring is closed.

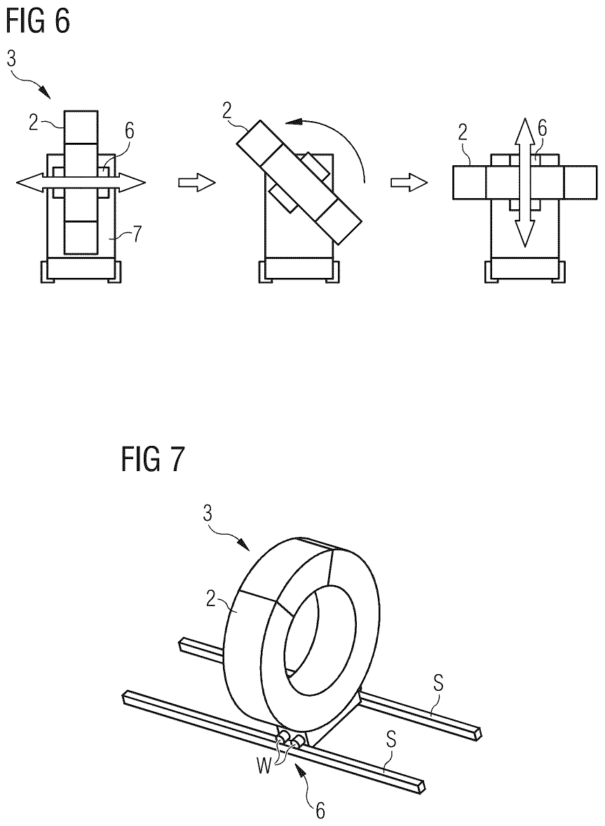

FIG. 6 shows a rotatable gantry apparatus on a trolley. In this example, the movement system 6 has a swivel joint to enable the gantry 2 to be tilted about an axis orthogonal to its main axis 8 using a motor. This enables the gantry to be moved from a position for examining a recumbent patient P (on the left) into a position for examining a standing patient P (on the right) via a simple rotation (center).

FIG. 7 shows a gantry 2 which can be moved on rails S via rollers W. The rails S and rollers W here represent the movement system 6. In this exemplary embodiment, the rails S are arranged on the floor of an examination room.

It is pointed out once again that the present invention described in detail above is merely a matter of exemplary embodiments which can be modified by the person skilled in the art in a wide variety of ways without departing from the scope of the present invention. Furthermore, the use of the indefinite articles “a” or “an” does not exclude the possibility that the features in question may be present more than once. Likewise, terms such as “unit” do not exclude the possibility that the components in question comprise several interacting sub-components, which may also be spatially distributed. The term “a number” is to be read as “at least one”.

Regardless of the grammatical gender of a particular term, persons with male, female or other gender identities are included.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A gantry apparatus for a CT system, the gantry apparatus comprising:
a movement system; and
a gantry including a plurality of radiation sources and radiation detectors, each radiation detector arranged on a plane around a main axis, wherein
the gantry is mounted to be moved laterally via the movement system, and
the movement system has guide elements and is configured to move the gantry in a straight line parallel to the main axis via the guide elements.

2. The gantry apparatus as claimed in claim 1, wherein
the movement system has, as the guide elements, at least two rails arranged parallel to each other, and includes movement elements connected to the gantry and configured to move along the at least two rails, or
the movement system has linear ball bearings with or in which the gantry is configured to slide.

3. The gantry apparatus as claimed in claim 1, wherein the movement system has a number of motors, and the movement system is configured to at least one of move the gantry along the main axis via a motor or rotate around the main axis.

4. The gantry apparatus as claimed in claim 1, wherein
the plurality of radiation sources and radiation detectors are arranged in a ring around the main axis, and
the gantry is configured to generate beams from several spatial directions through a center of the gantry onto the radiation detectors.

5. The gantry apparatus as claimed in claim 1, wherein the gantry has an open ring or a closed ring that is configured to open.

6. The gantry apparatus as claimed in claim 1, further comprising:
a robotic holding apparatus configured to move the gantry along non-linear trajectories in a room.

7. The gantry apparatus as claimed in claim 1, wherein the gantry apparatus has intrinsic X-ray protection.

8. The gantry apparatus as claimed in claim 1, further comprising:
a plurality of interfaces to integrate image and command transfer for external devices.

9. The gantry apparatus as claimed in claim 1, further comprising:
control elements for users.

10. The gantry apparatus as claimed in claim 1, wherein the movement system has a swivel joint to enable tilting of the gantry about an axis orthogonal to the main axis.

11. The gantry apparatus as claimed in claim 1, further comprising:
a handle with power sensors for power-assisted movement of the gantry.

12. The gantry apparatus as claimed in claim 1, wherein the movement system is configured to move the gantry in a direction orthogonal to the main axis.

13. The gantry apparatus as claimed in claim 1, further comprising:
a trolley configured to be locked for an examination, the trolley including a support apparatus for the gantry.

14. The gantry apparatus as claimed in claim 1, further comprising:
a battery system configured to supply power to the gantry; and
a radio system configured to wirelessly transmit image data.

15. A CT system comprising the gantry apparatus as claimed in claim 1.

16. The gantry apparatus of claim 2, wherein the movement elements are configured to move along the at least two rails at least one of in a sliding manner, via rollers or via balls.

17. The gantry apparatus of claim 4, wherein at least one of the plurality of radiation sources or the radiation detectors are arranged statically in the gantry apparatus.

18. The gantry apparatus of claim 5, wherein an opening of the gantry is greater than 60 cm.

19. The gantry apparatus of claim 18, wherein the opening of the gantry is greater than 80 cm.

20. The gantry apparatus as claimed in claim 7, wherein the intrinsic X-ray protection is in the form of a lead lining of the gantry.

21. The gantry apparatus as claimed in claim 8, wherein the plurality of interfaces are configured to integrate optical tracking, external software for image processing or an image display in a room.

22. The gantry apparatus as claimed in claim 10, wherein the movement system is configured to tilt the gantry via a motor.

23. The gantry apparatus as claimed in claim 11, further comprising:
a collision sensor, wherein the gantry apparatus is configured for sensor-supported collision avoidance or positioning.

24. The gantry apparatus as claimed in claim 12, wherein the movement system is configured to move the gantry in the direction orthogonal to the main axis via a motor.

25. The gantry apparatus as claimed in claim 13, wherein the trolley is an omnidirectional trolley.
